SEQUENCE LISTING (1 sequence)

SEQ ID NO: 1 - protein, 39 aa, Artificial Sequence
  source: /note=“Description of Artificial Sequence Synthetic polypeptide”
  MOD_RES (3)..(3): Alpha-Glu
  MOD_RES (9)..(9): Alpha-Asp
  MOD_RES (15)..(15): Alpha-Glu
  MOD_RES (16)..(16): Alpha-Glu
  MOD_RES (17)..(17): Alpha-Glu
  MOD_RES (24)..(24): Alpha-Glu
HGXGTFTSXLSKQMXXXAVRLFIXWLKNGGPSSGAPPPS

(19) United States
(12) Patent Application Publication
(10) Pub. No.: US 2022/0249618 A1
(43) Pub. Date: Aug. 11, 2022

(54) EXENATIDE COMPOSITIONS FOR PULMONARY ADMINISTRATION AND USE THEREOF

(71) Applicant: Aerami Therapeutics, Inc., Durham, NC (US)

(72) Inventors: Blaine BUECHE, San Mateo, CA (US); Mei-chang KUO, Palo Alto, CA (US); John PATTON, San Francisco, CA (US); Ian CHAN, South San Francisco, CA (US); Benjamin STEDMAN, San Jose, CA (US)

(21) Appl. No.: 17/621,379

(22) PCT Filed: Jul. 1, 2020
(86) PCT No.: PCT/US2020/040513

§ 371 Date: Dec. 21, 2021

Related U.S. Application Data
(60) Provisional application No. 62/870,447, filed on Jul. 3, 2019.

(51) Int. Cl. A61K 38/26; A61K 9/00; A61K 47/26; A61K 47/12

(52) U.S. Cl. CPC A61K 38/26; A61K 47/12; A61K 47/26; A61K 9/0078

(57) ABSTRACT

Provided herein are pharmaceutical compositions comprising exenatide and an aqueous buffer, wherein the pharmaceutical compositions are packaged for administration via inhalation. Methods for treating diabetes mellitus are also described.

### CROSS-REFERENCE TO RELATED APPLICATION

This application claims the benefit of and priority to U.S. Provisional Application No. 62/870,447, filed Jul. 3, 2019, which is incorporated herein by reference in its entirety.

### FIELD

The present disclosure is related to exenatide compositions. More particularly, the present disclosure relates to exenatide compositions for pulmonary administration.

### SEQUENCE LISTING

The official copy of the sequence listing is submitted electronically via EFS-Web as an ASCII formatted sequence listing with a file named 093088-1192592_(001810WO)_SL.txt, created on Jun. 30, 2020 and having a size of 1,168 bytes and is filed concurrently with the specification. The sequence listing contained in this ASCII formatted document is part of the specification and is herein incorporated by reference in its entirety.

### BACKGROUND

Diabetes mellitus is a metabolic disorder in which an individual's ability to moderate blood glucose levels in response to insulin is lost. Insulin is a hormone secreted by the pancreas into the blood that triggers cells to take up glucose. When the body cannot produce insulin, as occurs in type 1 diabetes, or is no longer responsive to insulin and/or produces less insulin, as occurs in type 2 diabetes, blood glucose levels rise. Complications from diabetes include increased risk of cardiovascular disease, neuropathy, nephropathy, retinopathy, foot damage, skin conditions, hearing impairment, and Alzheimer's disease. Treatment for type 1 diabetes involves insulin injections or the use of an insulin pump. Type 2 diabetes is also often treated with insulin injections or pumps. Exenatide subcutaneous injection is also currently approved to treat type II diabetes mellitus. However, currently available injected formulations provide limited dosing regiments (e.g., only one low dose or one high dose given twice daily) with almost no possibility for dose titration. The side effects of exenatide include nausea, upset stomach, vomiting and diarrhea especially when given at higher doses, which is often encountered with the limited dosing ranges of the currently available drug products. Lower doses of exenatide can lead to ineffective glucose control.

### BRIEF SUMMARY

Provided herein are pharmaceutical compositions comprising exenatide, or a pharmaceutically acceptable salt thereof, and an aqueous buffer, wherein the pharmaceutical compositions are packaged for administration via inhalation. In some embodiments, the pharmaceutical compositions are packaged for administration with a vibrating mesh device.

Also provided herein are methods for treating diabetes mellitus. The methods include administering a therapeutically effective amount of a pharmaceutical composition as described herein to a subject in need thereof, wherein the composition is administered to the subject via inhalation.

### BRIEF DESCRIPTION OF THE DRAWINGS

FIG. 1 shows the % assay recovery of the formulation at 4° C. analyzed by reverse-phase high performance liquid chromatography (RP-HPLC) using the ammonium bicarbonate method according embodiments of the present disclosure.

FIG. 2 shows the % total exenatide-related substances formed in the formulation at 4° C. analyzed by RP-HPLC using the ammonium bicarbonate method according embodiments of the present disclosure.

FIG. 3 shows the % assay recovery of the formulation at 25° C. analyzed by RP-HPLC using the ammonium bicarbonate method according embodiments of the present disclosure.

FIG. 4 shows the % total exenatide-related substances of the formulation at 25° C. analyzed by RP-HPLC using the ammonium bicarbonate method according embodiments of the present disclosure.

FIG. 5 shows the % assay recovery at 4° C. analyzed by RP-HPLC using the trifluoroacetic acid (TFA) method according embodiments of the present disclosure.

FIG. 6 shows the % exenatide-related substances of the formulation at 4° C. analyzed by RP-HPLC using the TFA method according embodiments of the present disclosure.

FIG. 7 shows the % assay recovery of the formulation at 25° C. analyzed by RP-HPLC using the TFA method according embodiments of the present disclosure.

FIG. 8 shows the % total exenatide-related substances of the formulation at 25° C. analyzed by RP-HPLC using the TFA method according embodiments of the present disclosure.

### DETAILED DESCRIPTION

The present disclosure provides liquid pharmaceutical compositions of the incretin mimetic exenatide. The compositions are suitable for inhalation, especially through a piezoelectric vibrating mesh inhalation device (sometimes referred to as a mesh nebulizer) that creates an aerosol suitable for deep lung inhalation. Deep lung inhalation, provided by the compositions and methods of the present disclosure, can deliver drugs efficiently into systemic blood circulation to treat diseases such as diabetes. Administration into the deep lung delivers the dose directly into the blood stream, and the compositions and methods described herein provide for improved titration of doses for patients with varying body mass and dose responses. Use of vibrating mesh nebulizers according to the methods of the present disclosure allows for titration of effective dosages with individual breaths. Dose titration using the compositions and methods of the present disclosure can minimize unwanted side effects and improve adherence in subjects who vary in weight and/or glycemic response.

### I. DEFINITIONS

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“Administering” or “administration of” a composition to a subject (and grammatical equivalents of this phrase), as used herein, refer to direct administration, which may be administration to a subject by a medical professional or may be self-administration, and/or indirect administration, which may be the act of prescribing a composition. For example, a physician who instructs a subject to self-administer a composition and/or provides a subject with a prescription for a composition is administering the composition to the subject.

“Comprising” is intended to mean that the compounds, compositions and methods include the recited elements, but does not exclude others. “Consisting essentially of” when used to define compounds, compositions and methods, shall mean excluding other elements that would materially affect the basic and novel characteristics of the claimed invention. Embodiments defined by each of these transition terms are within the scope of the present disclosure.

“Chemically stable” and “chemical stability,” as used herein, refers to the reactivity of exenatide in a pharmaceutical composition and the propensity of exenatide to react chemically, or decompose chemically, in the pharmaceutical composition. For example, a pharmaceutical composition is chemically stable when the total degradation products of exenatide remain below a limit about 10% of the sum of peak areas of all degradants, as calculated on a normalized peak area determined by high-performance liquid chromatography.

“Physical stability”, as used herein, refers to the ability of exenatide to retain its normal physical structure in a pharmaceutical composition and, as a result, the propensity of exenatide to not aggregate and/or precipitate out of solution during storage and usage. For example, the physical stability of a pharmaceutical composition may be reflected by the ability of the exenatide to retain its native configuration in the pharmaceutical composition.

“Pharmaceutically acceptable salt,” as used herein, refers to acid or base salts of exenatide. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid, fumaric acid, and the like) salts, and quaternary ammonium (methyl iodide, ethyl iodide, and the like) salts. By “pharmaceutically acceptable,” it is meant that the salt is compatible with the other ingredients of the composition and is not toxic or otherwise deleterious to the recipient thereof.

“Preservative,” as used herein, refers to a class of compounds that prevents or inhibits the growth of microorganisms, as well as compounds that help control oxidation reactions in pharmaceuticals. Phenol and meta-cresol are examples of preservatives.

“Surfactants,” as used herein, refers to amphiphilic organic compounds (having hydrophobic groups and hydrophilic groups) that aggregate to form micelles in aqueous compositions at critical concentrations, providing greater solubility for hydrophobic compounds. Surfactants may be applied to compositions may increase the physical stability of the compositions, modify their solubility, or both.

“Therapeutically effective amount” of a pharmaceutical composition, as used herein, refers to an amount of the composition that, when administered to a subject with diabetes mellitus, will have the intended therapeutic effect, for example, increased cellular uptake of blood glucose and reduced blood glucose levels. A therapeutically effective amount may be administered in one or more administrations.

“Treating” or “treatment of” a condition or subject, as used herein, refers to taking action to obtain beneficial or desired results, including clinical results, for a subject. For purposes of this disclosure, beneficial or desired clinical results include, but are not limited to, increased cellular uptake of blood glucose, reduced blood glucose levels, or both.

“About” and “around,” as used herein, indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. PHARMACEUTICAL COMPOSITIONS FOR ADMINISTRATION VIA INHALATION

Provided herein are pharmaceutical compositions comprising exenatide, or a pharmaceutically acceptable salt thereof, and an aqueous buffer, wherein the pharmaceutical compositions are packaged for administration via inhalation. Exenatide is also referred to as Exendin 4 and has the amino acid residue sequence: L-histidylglycyl-L-alpha-glutamylglycyl-L-threonyl-L-phenylalanyl-L-threonyl-L-seryl-L-alpha-aspartyl-L-leucyl-L-seryl-L-lysyl-L-glutaminyl-L-methionyl-L-alpha-glutamyl-L-alpha-glutamyl-L-alpha-glutamyl-L-alanyl-L-valyl-L-arginyl-L-leucyl-L-phenylalanyl-L-isoleucyl-L-alpha-glutamyl-L-tryptophyl-L-leucyl-L-lysyl-L-asparaginylglycylglycyl-L-prolyl-L-seryl-L-serylglycyl-L-alanyl-L-pro lyl-L-prolyl-L-prolyl-L-serinamide (SEQ ID NO:1).

The concentration of exenatide or pharmaceutically acceptable salt thereof may vary depending on factors including, but not limited to, the particular excipients employed in the pharmaceutical composition and the device to be used in the administration of the composition. In some embodiments, the concentration of exenatide ranges from about 200 μg/mL to about 800 μg/mL. The concentration of the exenatide may range, for example, from about 200 μg/mL to about 300 μg/mL, or from about 225 μg/mL to about 275 μg/mL, or from about 240 μg/mL to about 260 μg/mL. The concentration may range from about 200 μg/mL to about 250 μg/mL, or from about 250 μg/mL to about 300 μg/mL, or from about 300 μg/mL to about 350 μg/mL, or from about 350 μg/mL to about 400 μg/mL, or from about 400 μg/mL to about 450 μg/mL, or from about 450 μg/mL to about 500 μg/mL, or from about 500 μg/mL to about 550 μg/mL, or from about 550 μg/mL to about 600 μg/mL, or from about 600 μg/mL to about 650 μg/mL, or from about 650 μg/mL to about 700 μg/mL, or from about 700 μg/mL to about 750 μg/mL, or from about 750 μg/mL to about 800 μg/mL. In some embodiments, the concentration of the exenatide is about 250 μg/mL.

The pH of the pharmaceutical composition has been found to contribute to the stability of exenatide, as described in more detail below. The pH may vary on factors including, but not limited to, the concentration of exenatide and the other components present in the pharmaceutical composition. In some embodiments, the pH of the composition ranges from about 4.6 to about 5.2. The pH of the composition containing exenatide or pharmaceutically acceptable salt thereof, for example, may range from about 4.6 to about 5.0, or from about 4.7 to about 4.9. The pH of a composition containing exenatide may range, for example, from about 4.6 to about 4.7, from about 4.7 to about 4.8, or from about 4.8 to about 4.9, or from about 4.9 to about 5.0, or from about 5.0 to about 5.1, or from about 5.1 to about 5.2. In some embodiments, the composition contains exenatide and the pH is around 5.0. In some embodiments, the composition contains exenatide and the pH is around 4.8. In some embodiments, the pH remains stable over time (e.g., during storage at 4° C. or 25° C. for at least 6 months).

The aqueous buffers in the pharmaceutical compositions of the present disclosure will contain water and buffering agent, as well as optional components such as cosolvents, salts, chelators, or the like. Examples of suitable buffering agents include, but are not limited to, 2-(N-morpholino)ethane-sulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate monobasic, potassium phosphate dibasic, sodium phosphate monobasic, sodium phosphate dibasic, phosphate-buffered saline, sodium citrate, sodium acetate, sodium acetate trihydrate, and sodium borate. Examples of suitable salts include, but are not limited to, NaCl, KCl, CaCl₂, and salts of Mn²⁺ and Mg²⁺.

In some embodiments, the aqueous buffer comprises acetate. The aqueous buffer may contain, for example, a sodium acetate buffer or an ammonium acetate buffer. In some embodiments, the aqueous buffer comprises sodium acetate. The concentration of the buffering agent (e.g., sodium acetate) may vary depending on factors including, but not limited to, the concentration of exenatide or the device to be used in the administration of the composition.

In some embodiments, the concentration of the buffering agent (e.g., sodium acetate) ranges from about 5 mM to about 50 mM. The concentration of the buffer may range, for example, from about 5 mM to about 25 mM, or from about 5 mM to about 20 mM, or from about 5 mM to about 15 mM, or from about 8 mM to about 12 mM. The concentration of the buffering agent may range from about 5 mM to about 10 mM, or from about 10 mM to about 20 mM, or from about 20 mM to about 30 mM, or from about 30 mM to about 40 mM to about 50 mM. In some embodiments, the aqueous buffer contains the buffering agent (e.g., sodium acetate) at a concentration ranging from about 5 mM to about 15 mM (e.g., about 10 mM).

In some embodiments, the osmolarity of composition ranges from about 50 mOsm to about 400 mOsm. The osmolarity of the composition may range, for example, from about 75 mOsm to about 375 mOsm, or from about 100 mOsm to about 350 mOsm, or from about 125 mOsm to about 325 mOsm, or from about 125 mOsm to about 300 mOsm, or from about 125 mOsm to about 275 mOsm, or from about 125 mOsm to about 250 mOsm, or from about 150 mOsm to about 225 mOsm, or from about 150 mOsm to about 200 mOsm, or from about 150 mOsm to about 175 mOsm, or from about 150 mOsm to about 170 mOsm, or from about 155 mOsm to about 165 mOsm. In some embodiments, the osmolarity of the composition is around 160 mOsm. Buffering agents, salts, and the like will contribute to the total osmolarity of the pharmaceutical compositions, and other agents such as dextrose, glycerin, mannitol, sucrose, and the like can be added to further adjust the osmolarity of the pharmaceutical composition. In some embodiments, the pharmaceutical composition additionally contains mannitol. In some embodiments, the concentration of mannitol ranges from about 50 mM to about 200 mM. The concentration of the mannitol may range, for example, from about 50 mM to about 190 mM, or from about 60 mM to about 180 mM, or from about 70 mM to about 170 mM, or from about 80 mM to about 160 mM, or from about 90 mM to about 150 mM, or from about 130 mM to about 160 mM, or from about 130 mM to about 150 mM, or from about 135 mM to about 145 mM. In some embodiments, the concentration of mannitol in the pharmaceutical composition is about 140 mM.

In some embodiments, the pharmaceutical composition is substantially free of preservatives. By “substantially free,” it is meant that the total concentration of preservative(s) in the pharmaceutical composition is equal to or less than 0.25% (w/w). In some embodiments, the total concentration of preservative(s) is less than 0.1% (w/w), less than 0.01% (w/w), less than 0.001% (w/w), or less than 0.0001% (w/w). In some embodiments, the total concentration of preservative(s) is 0% (w/w). In some instances, the preservatives are phenolic compounds. Examples of phenolic compounds include phenol, cresol, and derivatives thereof. In some instances, the compositions do not contain organic solvents. In certain aspects, the compositions do not contain alcohols, including polyols, sugars, amino acids, or amines. Compositions containing preservatives are described, for example, in U.S. Pat. Nos. 6,489,292 and 6,211,144, which are incorporated herein by reference. Such preservatives can include phenol and derivatives thereof such as meta-cresol, chloro-cresol, methylparaben, ethyl paraben, propyl paraben, thymol, as well as derivatives thereof and mixtures of such compounds. Some similar non-phenol preservatives include bi- or tricyclic aliphatic alcohols and purines, such as a bicyclic aliphatic alcohol, including a monoterpenol, such as isopinocampheol, 2,3-pinandiol, myrtanol, bomeol, norbomeol or fenchol, a tricyclic aliphatic alcohol, such as 1-adamantanol, and purines such as adenine, guanine or hypoxanthine. Other exemplary preservatives include sodium benzoate, benzalkonium chloride, benzyl alcohol, and thimerosal. Such preservatives are generally included to ensure stability or sterility of pharmaceutical compositions. In contrast, the compositions of the present disclosure maintain stability and/or or sterility without including preservatives. In some instances, the compositions do not contain phenol, cresol, or derivatives of either.

In some embodiments, the compositions do not contain surfactants. For example, amphipathic excipients that modify the surface tension between a solution and any interface (for example, a liquid/glass vial interface, an air/liquid interface) may be excluded from the compositions. Surfactants such as polysorbate-80 and Triton™ X-100 are well-known excipients, but they may in some instances cause foaming and loss of physical stability upon nebulization or aerosolization. As such, the compositions of the present disclosure provide an advantage over compositions containing surfactants.

Surprisingly, the pharmaceutical compositions described herein possess chemical stabilities (as measured by the extent of drug degradation over time) that are equivalent to or greater than conventional pharmaceutical compositions that include undesirable additives. In particular, the chemical stability of the pharmaceutical composition described herein is achieved absent the addition of solubility enhancers (other than co-solvent), surfactant addition, incorporation of stabilizers, incorporation of dispersants, and other such similar approaches that often involve the use of materials considered to be undesirable for direct delivery to lung tissue. Advantageously, the pharmaceutical composition achieves both a low rate and low degree of chemical degradation over time.

In some embodiments, the chemical stability of the pharmaceutical composition under storage conditions of 4° C. for a period up to 6 months is greater than 95% as measured by RP-HPLC using trifluoroacetic acid as a mobile-phase additive, e.g., greater than 95%, greater than 96%, greater than 97%, greater than 98%, or greater than 99%. In some embodiments, the chemical stability of the pharmaceutical composition under storage conditions of 25° C. for a period up to 6 months is greater than 90% as measured by RP-HPLC using trifluoroacetic acid as a mobile-phase additive, e.g., greater than 91%, greater than 92%, greater than 93%, greater than 94%, or greater than 95%. In some embodiments, the chemical stability of the pharmaceutical composition under storage conditions of 4° C. for a period up to 2 months is greater than 92% as measured by RP-HPLC using trifluoroacetic acid as a mobile-phase additive, e.g., greater than 93%, greater than 94%, greater than 95%, greater than 96%, greater than 97%, greater than 98%, or greater than 99%. In some embodiments, for storage conditions at 4° C. and/or 25° C., degradants (e.g., exenatide-related substances) may be produced at a low rate in the provided composition, resulting in an extended period of stability.

Generally, the pharmaceutical composition provided herein has an extended shelf life, where shelf life is characterized by a degree of chemical degradation of exenatide of no greater than 10% during a 6 month period. In some embodiments, the extent of chemical degradation of the pharmaceutical composition under storage conditions after 6 months is less than 10%, e.g., less than 9%, or less than 8%, less than 7%, less than 6%, or less than even 5%. As described in Example 6, degradants (e.g., exenatide-related substances) were produced at a low rate, even under accelerated storage conditions of 25° C., which indicates that the pharmaceutical composition can achieve prolonged periods of stability.

In some embodiments, the pharmaceutical composition consists essentially of exenatide, or a pharmaceutically acceptable salt thereof, and an aqueous buffer.

In some embodiments, the pharmaceutical composition comprises exenatide or pharmaceutically acceptable salt thereof in an amount ranging from about 250 μg/ml to about 350 μg/ml; the pH of the aqueous buffer ranges from about 4.7 to about 4.9; the osmolarity of the composition ranges from about 150 mOsm to about 200 mOsm; and the composition is substantially free of preservatives.

In some embodiments, the pharmaceutical composition comprises exenatide or pharmaceutically acceptable salt thereof in an amount ranging from about 250 μg/ml to about 350 μg/ml; the pH of the aqueous buffer ranges from about 4.7 to about 4.9; the osmolarity of the composition ranges from about 150 mOsm to about 200 mOsm; and the composition is substantially free of preservatives and surfactants.

Pharmaceutical compositions of the present disclosure may be packaged as a single use “unit dose” container or as a multi-dose container. In some instances, a unit dose of the compositions described in this disclosure is provided. Examples of single use containers are blister packs or capsules. Examples of multi-dose containers are drop dispensers, or vials. Kits according to the present disclosure may include one or more unit doses of a composition and a device for administering the composition. Kits may include a single use “unit dose” container or a multi-dose container. Examples of single use containers are blister packs or capsules. Examples of multi-dose containers are drop dispensers, or vials. In some instances, the device for administering the composition may be an aerosolization device. For example, in some instances, the device may be an aerosolizer, an inhaler, or a nebulizer. Exemplary devices that may be included in the kit are described in U.S. Pat. Nos. 8,950,394 and 10,307,550; U.S. Pat. Appl. Pub. Nos. 2013/0269684, 2013/0269694, 2013/0269684, 2015/0352301, 2016/0001018, and 2016/0001019; and International PCT Publication Nos. WO 2013/158352 and WO 2013/158353, each of which is incorporated herein by reference in its entirety. Other devices for aerosolization of liquid compositions are well-known in the art. In some instances, the kits may include a device for administrating the composition via injection. For example, the kits may include one or more syringes. In another example, the kits may include one or more needles. In another example, the kits may include one or more syringes and one or more needles. The kits may also include a pump or a pen device for administering the composition via injection. In some instances, the kit may include instructions describing use of the device to administer the composition.

In some embodiments, the pharmaceutical composition can be aerosolized, as described further below. In some embodiments, the pharmaceutical composition can be aerosolized using a vibrating mesh inhaler. The particle size Dv 50 (equivalent to mass medium aerodynamic diameter or MMAD) of the aerosolized pharmaceutical composition may range from 0.5 μm to 25 μm as measured using a Malvern Mastersizer laser diffraction instrument, e.g., 1 μm to 20 μm, 1.5 μm to 15 μm, 2 μm to 12 μm, 2.5 μm to 10 μm, 3 μm to 8 μm, or 4 μm to 6 μm.

In some embodiments, the pharmaceutical composition described herein achieves an emitted dose from an inhaler that improves delivery to the lungs of a subject. In some embodiments, the emitted dose of the pharmaceutical composition from an inhaler, as measured by HPLC, may be greater than 75%, e.g., greater than 76%, greater than 77%, greater than 78%, greater than 79%, greater than 80%, greater than 81%, greater than 82%, greater than 83%, greater than 84%, greater than 85%, greater than 86%, greater than 87%, or greater than 88%. In certain aspects, a residual amount of the pharmaceutical composition deposited in the inhaler is significantly limited due to the favorable aerosolization properties of the pharmaceutical composition. In some embodiments, the residual amount of the pharmaceutical composition deposited in the inhaler is less than 20%, e.g., less than 18%, less than 17%, less than 16%, less than 15%, less than 14%, less than 13%, or less than 12%. Therefore, the pharmaceutical composition can be effectively aerosolized and delivered to the lungs of a subject.

### III. METHODS FOR AEROSOLIZATION AND TREATMENT OF DIABETES MELLITUS

Provided herein are methods for treating diabetes mellitus using the described pharmaceutical compositions. The methods include administering a therapeutically effective amount of a pharmaceutical composition as described herein to a subject in need thereof, wherein the composition is administered to the subject via inhalation. For example, the composition can be administered using an inhalation device such as an aerosolizer, an inhaler, or a nebulizer, or by injection (intravenous, intramuscular, intraperitoneal), including by pump or pen. In some embodiments, methods include the use of a pharmaceutical composition which is packaged in a dispenser for administration via inhalation in conjunction with a vibrating mesh nebulizer. Also provided herein are methods of aerosolizing the described pharmaceutical compositions.

Exemplary devices for aerosolizing and administering the provided preservative free compositions are described in U.S. Patent Application Publication Nos. 20110168172; 20110017431; 20130269684; 20130269694; and 20130269684; U.S. application Ser. Nos. 14/743,763; 14/743,711; 14/732,247; and Ser. No. 14/732,446; and International PCT Publication Nos. WO 2013/158352 and WO 2013/158353, each of which is incorporated herein by reference in their entirety. Other devices for aerosolization of liquid compositions such as those described herein are well-known in the art.

In some embodiments, the composition is administered prior to the subject eating a meal. For example, the composition may be administered just prior to the subject eating a meal. Alternatively, the composition may be administered at least 15 minutes prior to the subject eating a meal. In some embodiments, the composition is administered at least once a day. In some embodiments, the composition is administered 1, 2, 3, or more times per day.

As mentioned above, side effects of exenatide (e.g., nausea) can result from the limited dosing regimens afforded by currently available injectable formulations. As shown in Table 1, the available dosing regimens are generally not adjustable for the body mass of an individual patient or the individual patient's response (e.g., to a dose under particular conditions).

TABLE 1
Available dosing regimens for injectable exenatide.

| | Formulation | Injection Size | Total dose |
|---|---|---|---|
| Dosage | (μg/mL) | (μL) | (2X per day) |
| 5 μg | 250 | 20 | 10 μg |
| 10 μg | 250 | 40 | 20 μg |

In contrast, Table 2 and Table 3 demonstrate the remarkable flexibility provided by pulmonary administration using the compositions and methods of the present disclosure. The use of the device with a composition and dispenser that delivers approximately 55-μL drops to the nebulizer improves the ability to titrate doses for patients with varying body mass and dose responses. Additionally, the dispenser can be modified to deliver even finer 25-μL drops, thus creating more flexibility. This allows for varying of drops/dosage prior to each meal (for example, 2 drops before breakfast, 3 drops before lunch and 4 drops before dinner), which can decrease the incidence of side effects such as nausea, increase patient compliance, and improve glucose control.

TABLE 2
Comparison of b.i.d. dosing options for injectable
and inhaled exenatide compositions.

| | Number of | Volume (mL) of | | | |
|---|---|---|---|---|---|
| Body | Drops | Composition | Total dose | Lung | Injected |
| Mass | (55 μL/drop) | (300 μg/mL) | delivered | dose | Dose |
| (kg) | per Dose | per Dose | (μg) | (μg)* | (μg) |
| 20 | 1 | 0.055 | 16.5 | 2.0 | 10 |
| 40 | 2 | 0.110 | 33.0 | 4.0 | 10 |
| 59 | 3 | 0.165 | 49.5 | 5.9 | 10 |
| 79 | 4 | 0.220 | 66.0 | 7.9 | 10 |
| 99 | 5 | 0.275 | 82.5 | 9.9 | 10 |
| 119 | 6 | 0.330 | 99.0 | 11.9 | 10 |

*Assuming 12% bioavailability upon inhalation (vs. subcutaneous injection) and adjusted based on body mass at 0.1 μg/kg per day

TABLE 3
Comparison of t.i.d. dosing options for injectable
and inhaled exenatide compositions.

| | Number of | Volume (mL) | | | |
|---|---|---|---|---|---|
| Body | Drops | of Composition | Total dose | Lung | Injected |
| Mass | (55 μL/drop) | (300 μg/mL) | delivered | dose | Dose |
| (kg) | per Dose | per Dose | (μg) | (μg) | (μg) * |
| 30 | 1 | 0.055 | 16.5 | 2.0 | 6.7 |
| 59 | 2 | 0.110 | 33.0 | 4.0 | 6.7 |
| 89 | 3 | 0.165 | 49.5 | 5.9 | 6.7 |
| 119 | 4 | 0.220 | 66.0 | 7.9 | 6.7 |
| 149 | 5 | 0.275 | 82.5 | 9.9 | 6.7 |
| 178 | 6 | 0.330 | 99.0 | 11.9 | 6.7 |

* The 6.7 μg per dose via injection is not possible with currently available needle injectors.
** Assuming 12% bioavailability upon inhalation (vs. subcutaneous injection) and adjusted based on body mass at 0.1 μg/kg per day.

In some embodiments, administering the pharmaceutical composition according to the methods of the present disclosure comprises aerosolizing one to six drops of the pharmaceutical composition. In some embodiments, the volume of each drop ranges from about 20 μL to about 60 μL. The volume of each drop may be, for example, around 25, 30, 35, 40, 45, 50, or 55 μL. Lung doses delivered by the methods of the invention may range, for example, from about 0.5 μg to about 20 μg (e.g., about 1-15 μg, or about 2-12 μg) and can be titrated with individual breathes (e.g., with 1 breath, or with 2-3 breaths, or with 3-4 breaths, or with 4-5 breaths) as described herein.

Compositions according to the present disclosure exhibit advantageous liquid output rates when used with vibrating mesh devices. Compositions which are substantially free of preservatives such as meta-cresol provide liquid output rates that are particularly advantageous for ensuring that the desired dose of exenatide or pharmaceutically acceptable salt thereof is delivered in one to three breaths. Typically, compositions according to the present disclosure will exhibit liquid output rates in excess of 325 μL/min when used with a vibrating mesh inhaler as described, for example, in US 2014/0318533 A1 that is actuated by a draw rate that approximated continuous breathing (around 10 L/min). In some embodiments, the exenatide is present in an amount ranging from about 280 μg/mL to about 600 μg/mL, and administering the composition comprises aerosolizing the composition at a rate ranging from 300 μL/min to about 700 μL/min. The liquid output rate for a particular composition can be measured and expressed as an absolute value, or as a relative value compared to a standard composition such as a sodium chloride solution. In some embodiments, for example, administering the composition includes aerosolizing the composition, and wherein the rate of aerosolization of the composition is around 0.4 to 1.1 times the rate of aerosolization of 140 mM NaCl.

### IV. EMBODIMENTS

The following examples are offered to illustrate, but not to limit, the claimed invention.

Embodiment 1: A pharmaceutical composition comprising exenatide, or a pharmaceutically acceptable salt thereof, and an aqueous buffer, wherein the pharmaceutical composition is packaged for administration via inhalation.

Embodiment 2: A pharmaceutical composition consisting essentially of exenatide, or a pharmaceutically acceptable salt thereof, and an aqueous buffer, wherein the pharmaceutical composition is packaged for administration via inhalation.

Embodiment 3: A pharmaceutical composition comprising exenatide, or a pharmaceutically acceptable salt thereof, and an aqueous buffer, wherein the pharmaceutical composition is packaged for administration via inhalation, wherein the pharmaceutical composition is substantially free of preservatives and/or surfactants.

Embodiment 4: An embodiment of any preceding or subsequent embodiment, wherein the pharmaceutical composition is substantially free of preservatives.

Embodiment 5: An embodiment of any preceding or subsequent embodiment, wherein the pharmaceutical composition is substantially free of surfactants.

Embodiment 6: An embodiment of any preceding or subsequent embodiment, wherein the exenatide or pharmaceutically acceptable salt thereof is present in an amount ranging from about 250 μg/ml to about 350 μg/ml; the pH of the aqueous buffer ranges from about 4.7 to about 4.9; and the osmolarity of the composition ranges from about 150 mOsm to about 200 mOsm.

Embodiment 7: An embodiment of any preceding or subsequent embodiment, wherein the concentration of exenatide or the pharmaceutically acceptable salt thereof ranges from about 200 μg/mL to about 800 μg/mL.

Embodiment 8: An embodiment of any preceding or subsequent embodiment, wherein the pH of the composition ranges from about 4.6 to about 5.2.

Embodiment 9: An embodiment of any preceding or subsequent embodiment, wherein the pH is about 4.8.

Embodiment 10: An embodiment of any preceding or subsequent embodiment, wherein the aqueous buffer comprises acetate.

Embodiment 11: An embodiment of any preceding or subsequent embodiment, wherein the aqueous buffer comprises sodium acetate.

Embodiment 12: An embodiment of any preceding or subsequent embodiment, wherein the concentration of sodium acetate ranges from about 5 mM to about 50 mM.

Embodiment 13: An embodiment of any preceding or subsequent embodiment, wherein the osmolarity of composition ranges from about 50 mOsm to about 400 mOsm.

Embodiment 14: An embodiment of any preceding or subsequent embodiment, wherein the pharmaceutical composition additionally comprises mannitol.

Embodiment 15: An embodiment of any preceding or subsequent embodiment, wherein the concentration of mannitol ranges from about 50 mM to about 200 mM.

Embodiment 16: An embodiment of any preceding or subsequent embodiment, wherein the pharmaceutical composition is substantially free of preservatives, stabilizers, and/or surfactants.

Embodiment 17: An embodiment of any preceding or subsequent embodiment, the exenatide or the pharmaceutically acceptable salt thereof is present in an amount ranging from about 250 μg/ml to about 350 μg/ml; the pH of the aqueous buffer is ranges from about 4.7 to about 4.9; the osmolarity of the composition ranges from about 150 mOsm to about 200 mOsm; and the composition is substantially free of preservatives

Embodiment 18: An embodiment of any preceding or subsequent embodiment, wherein the pharmaceutical composition is packaged in a dispenser for administration via inhalation using a vibrating mesh nebulizer.

Embodiment 19: An embodiment of any preceding or subsequent embodiment, wherein the composition comprises exenatide acetate.

Embodiment 20: A method of treating a subject with diabetes mellitus, comprising administering a therapeutically effective amount of the pharmaceutical composition according to any preceding or subsequent embodiment, wherein the composition is administered to the subject via inhalation.

Embodiment 21: An embodiment of any preceding or subsequent embodiment, wherein the composition is administered using a vibrating mesh nebulizer.

Embodiment 22: An embodiment of any preceding or subsequent embodiment, wherein the therapeutically effective amount of the pharmaceutical composition is administered in one to five breaths.

Embodiment 23: An embodiment of any preceding or subsequent embodiment, wherein the pharmaceutical composition is administered two times per day or three times per day.

Embodiment 24: An embodiment of any preceding or subsequent embodiment, wherein administering the pharmaceutical composition comprises aerosolizing one to six drops of the pharmaceutical composition.

Embodiment 25: An embodiment of any preceding or subsequent embodiment, wherein the volume of each drop ranges from about 20 μL to about 60 μL.

Embodiment 26: An embodiment of any preceding or subsequent embodiment, wherein the exenatide or the pharmaceutically acceptable salt thereof is present in an amount ranging from about 200 μg/mL to about 800

Embodiment 27: An embodiment of any preceding or subsequent embodiment, wherein around 1-15 μg of exenatide or a pharmaceutically acceptable salt thereof are delivered to the lungs of the subject in each administration.

Embodiment 28: An embodiment of any preceding or subsequent embodiment, wherein the composition has a chemical stability of at least 95% for 6 months under storage conditions of 4° C.

### V. EXAMPLES

The following examples are offered to illustrate, but not to limit, the claimed subject matter.

### Example 1. Study of Liquid Output Rate

The liquid output rate of exenatide compositions according to the present disclosure, measured according to the procedure described below, demonstrates the compatibility of the compositions with vibrating mesh nebulizers. When used with the compositions according to the present disclosure (e.g., Composition 1 in Table 4), the nebulizer created fine particles for introduction of the delivered dose to the lung in a minimal number of breaths. In contrast, compositions containing phenolic preservatives (e.g., Composition 2 in Table 4) were low performing and did not move liquid through the nebulizer efficiently.

TABLE 4

| | | | Sodium | | Preservative | Total |
|---|---|---|---|---|---|---|
| | Exenatide | Exenatide | Acetate | Mannitol | (meta-cresol) | Osm |
| Composition | (mg/mL) | (mM) | (mM) | (mM) | (mM) | (mOsm) |
| Composition 1 | 0.3000 | 0.072 | 10.0 | 140.0 | 0.0 | 160.2 |
| Composition 2 | 0.2500 | 0.060 | 10.0 | 260.0 | 20.3 | 300.5 |

Liquid output rate was measured with a breath-actuated vibrating mesh device. The device reservoir/mouthpiece (described in US 2014/0318533 A1) was placed on a microbalance and tared. The reservoir/mouthpiece was filled with 200 μL of solution using a calibrated pipette, and the reservoir/mouthpiece was weighed again to record the amount of solution present. The device was equipped with silicone tubing connected to a vacuum pump to simulate continuous patient breathing at 10 L per minute. The pump was started and timed, in seconds, while monitoring the liquid in reservoir. Timing was stopped when liquid was no longer emitted and the reservoir was empty. The reservoir/mouthpiece was weighed again to determine the amount of remaining liquid. The output volume was calculated by subtracting the residual mass from the original mass; the volume of the solution is equivalent to mass since the density of the tested compositions is 1.0 g per 1.0 mL. The mass/output volume was divided by output time to calculate the liquid output rate, which is reported in μL/min. The measured liquid output rates for various compositions are shown in Table 5.

TABLE 5

| | Starting | | | | Average |
|---|---|---|---|---|---|
| | Dose | Residual | Aerosolization | Aerosolization | Aerosolization |
| | Weight | Dose | Time | Rate | Rate |
| Run Information | (mg) | (mg) | (seconds) | (μL/min) | (μL/min) |
| 140 mM NaCl a | 201.92 | 43.68 | 14 | 678.2 | 647.4 |
| 140 mM NaCl b | 202.04 | 46.72 | 15 | 621.3 | |
| 140 mM NaCl c | 202.25 | 41.59 | 15 | 642.6 | |
| Composition 1, run a | 202.40 | 30.5 | 18 | 558.1 | 559.2 |
| Composition 1, run b | 206.60 | 28.2 | 19 | 557.2 | |
| Composition 1, run c | 206.00 | 30.1 | 19 | 562.3 | |
| Composition 2, run a | 200.46 | 14.99 | 35 | 317.9 | 319.3 |
| Composition 2, run b | 201.12 | 14.11 | 35 | 320.6 | |

### Example 2. Study of Chemical Stability

Several aqueous solutions containing exenatide were screened for stability using reverse phase HPLC employing a C18 column in a procedure based on the USP monograph's “Exenatide related substances and impurities.” The stability screening experiment was performed by stressing approximately 5 mL of each formulation in borosilicate glass vials with Teflon-lined caps at 25° C. for 4 weeks. The stability results are summarized in Table 6A and Table 6B. Sodium acetate provided the best chemical stability as compared to sodium citrate and 70 mM sodium chloride. Surprisingly, citrate buffer produced notably less stability. Furthermore, buffer solutions at pH 4.8-5.0 provided better exenatide stability than buffer solutions at pH 4.5, minimizing formation of three primary degradation products. Compositions of the present disclosure, characterized by low osmolarity and the absence of phenolic preservatives such as m-cresol, improved the chemical stability of exenatide. Compositions containing 5-10 mM sodium acetate buffer, 140 mM mannitol, and an overall osmolarity of 160-180 mOsm, provided exceptional exenatide stability and compatibility with the breath-actuated vibrating mesh inhaler.

TABLE 6A
Results of exenatide (exe.) stability screening at pH 4.5 with different buffers.

| | Exe. | Deg 1 | Deg 2 | Deg 3 | Deg 4 | Deg 6 | Deg 7 | Deg 8 | Deg 9 |
|---|---|---|---|---|---|---|---|---|---|
| | (%) | (%) | (%) | (%) | (%) | (%) | (%) | (%) | (%) |
| Citrate | 95.06 | 0.11 | 0.05 | 0.07 | 0.28 | 0.07 | 0.57 | 0.23 | 1.7 |
| Acetate | 96.23 | 0.05 | 0.03 | 0.11 | 0.28 | 0.15 | 0.17 | 0.22 | 0.87 |
| NaCl | 95.12 | 0.04 | 0.04 | 0.18 | 0.45 | 0.36 | 0.40 | 0.23 | 1.25 |
| Table 4, # | 96.66 | ND | 0.02 | ND | ND | 0.12 | 0.32 | 0.28 | |
| 2 | | | | | | | | | |

TABLE 6B
Results of exenatide (exe.) stability screening at pH 5.0 with different buffers.

| | Exe. | Deg 1 | Deg 2 | Deg 3 | Deg 4 | Deg 6 | Deg 7 | Deg 8 | Deg 9 |
|---|---|---|---|---|---|---|---|---|---|
| | (%) | (%) | (%) | (%) | (%) | (%) | (%) | (%) | (%) |
| Citrate | 96.11 | 0.08 | 0.08 | 0.06 | 0.18 | 0.07 | 0.35 | 0.13 | 1.17 |
| Acetate | 96.99 | 0.02 | 0.07 | 0.07 | 0.18 | 0.13 | 0.43 | 0.14 | 0.54 |
| NaCl | 96.02 | 0.02 | 0.11 | 0.18 | 0.31 | 0.25 | 0.17 | 0.14 | 1.02 |

### Example 3. Study of Aerosol Formation

The aerosol particle size of the microdroplets produced with compositions of the present disclosure by the vibrating mesh inhaler was measured using the Malvern Mastersizer laser diffraction instrument. The measured particle size Dv 50 (equivalent to mass medium aerodynamic diameter or MMAD) was 4.4 μm. As described above for the measurement of liquid output rate, the inhaler device was attached to the laser diffraction instrument and a pump rate of 10 L/min was employed to activate the device, generating aerosol particles that were directed into the laser path. The laser diffraction measurements verify that very small uniform particles are generated, which are suitable for deep lung deposition.

### Example 4. Study of Emitted Dose of Aerosol Formation

The emitted dose of aerosol produced from the inhalation formulations of the present disclosure were investigated. The emitted dose (ED) of the formulation was tested using the AFINA Inhaler™ (from Aerami Therapeutics) with three different mouthpieces (MPCs) (same design) for three replicates of the formulation.

The formulations comprised approximately 0.3 mg/mL exenatide, 1.36 mg/mL sodium acetate trihydrate, and 25.5 mg/mL mannitol at a pH 4.8. Standards were made with USP Exenatide Reference Standard diluted to a range of 0.45 μg/mL to 15 μg/mL in 70/30/0.1 water/acetonitrile/Tween® 20 (Diluent 1) to encompass the concentration of the ED samples. Each ED replicate was 220 μL and was aerosolized into a Respirgard II™ filter. Each MPC was weighed at the following time points: 1) before loading the formulation; 2) with the loaded dose of the formulation; and 3) after aerosolization of the formulation to determine residual in the MPC. Samples were then extracted using 2 mL of acetonitrile (Diluent 2) pipetted into the Respirgard II™ filter followed by adding 13 mL of 80/20/0.1% water/acetonitrile/Tween® 20 (Diluent 3). The Respirgard II™ filter was then capped and agitated by hand for 60 seconds. The MPC was placed into a bag with 5 mL of Diluent 1 and agitated by hand for 60 seconds. The resulting samples were analyzed by HPLC (J.T. Baker). The extracted filter samples were analyzed neat by HPLC. The replicates were compared to the generated linear calibration curve (y=141220.16x−18827.17; R²=0.9999). The area response was compared to the linear calibration curve to determine overall ED and residual in the MPC. The % residual, % ED, and % mass balance (MB) were determined using the balance weight of the residual (Table 7) and by HPLC assay (Table 8).

TABLE 7
Overall Emitted Dose and MPC Residual - Balance Weight of Residual

| | | Starting Dose | Residual Dose | % | Emitted Dose | % ED |
|---|---|---|---|---|---|---|
| | | Weight (mg) | in MPC (mg) | Residual | (mg) | (wt. %) |
| MPC 1 | ED 1 | 216.59 | 27.07 | 12.5 | 189.5 | 87.5 |
| | ED 2 | 220.15 | 26.22 | 11.9 | 193.9 | 88.1 |
| | ED 3 | 221.32 | 28.77 | 13.0 | 192.6 | 87.0 |
| | | | Avg. | 12.5 | 192.0 | 87.5 |
| | | | STD | 0.5 | 2.3 | 0.5 |
| MPC 2 | ED 1 | 219.56 | 28.82 | 13.1 | 190.7 | 86.9 |
| | ED 2 | 212.63 | 28.66 | 13.5 | 184.0 | 86.5 |
| | ED 3 | 221.49 | 28.88 | 13.0 | 192.6 | 87.0 |
| | | | Avg. | 13.2 | 189.1 | 86.8 |
| | | | STD | 0.2 | 4.5 | 0.2 |
| MPC 3 | ED 1 | 224.61 | 10.72 | 4.8 | 213.9 | 95.2 |
| | ED 2 | 219.06 | 12.33 | 5.6 | 206.7 | 94.4 |
| | ED 3 | 220.07 | 11.23 | 5.1 | 209.5 | 94.9 |

| | | | |
|---|---|---|---|
| Avg. | 5.2 | 210.1 | 94.8 |
| STD | 0.4 | 3.6 | 0.4 |
| Avg. (n = 9) | 10.3 | 197.1 | 89.7 |
| STD (n = 9) | 3.9 | 10.3 | 3.9 |
| % RSD | 37.6 | 5.2 | 4.3 |

TABLE 8
Overall Emitted Dose, MPC Residual, and % Mass Balance - HPLC

| | | Nominal | Residual | % | Emitted | % ED | |
|---|---|---|---|---|---|---|---|
| | | Dose (mg) | Dose (μg) | Residual | Dose (μg) | (wt. %) | % MB |
| MPC 1 | ED 1 | 60.9 | 11.0 | 18.4 | 49.4 | 82.4 | 100.9 |
| | ED 2 | 60.9 | 11.1 | 16.8 | 51.0 | 83.7 | 100.5 |
| | ED 3 | 60.9 | 11.4 | 17.1 | 51.9 | 84.7 | 101.8 |
| | | | Avg. | 17.5 | 50.8 | 83.6 | 101.1 |
| | | | STD | 0.8 | 1.2 | 1.1 | 0.7 |
| MPC 2 | ED 1 | 60.9 | 11.0 | 16.8 | 49.0 | 80.7 | 97.5 |
| | ED 2 | 60.9 | 11.3 | 17.8 | 48.5 | 82.4 | 100.2 |
| | ED 3 | 60.9 | 12.6 | 19.0 | 50.7 | 82.7 | 101.7 |
| | | | Avg. | 17.8 | 49.4 | 81.9 | 99.8 |
| | | | STD | 1.1 | 1.2 | 1.1 | 2.1 |
| MPC 3 | ED 1 | 60.9 | 5.4 | 8.1 | 54.6 | 87.9 | 95.9 |
| | ED 2 | 60.9 | 6.2 | 9.5 | 54.4 | 89.8 | 99.3 |
| | ED 3 | 60.9 | 6.6 | 10.0 | 53.7 | 87.9 | 97.9 |

| | | | | |
|---|---|---|---|---|
| Avg. | 9.2 | 54.3 | 88.5 | 97.7 |
| STD | 1.0 | 0.5 | 1.1 | 1.7 |
| Avg. | 14.8 | 51.5 | 84.7 | 99.5 |
| (n = 9) | | | | |
| STD | 4.3 | 2.3 | 3.1 | 2.0 |
| (n = 9) | | | | |
| % RSD | 29.2 | 4.5 | 3.7 | 2.0 |

The actual concentration of the formulation was determined to be 0.277 mg/mL. As shown in Table 7, based the weight of the MPC before and after, there was an average residual dose in the mouthpiece of 10.3%+3.9%. Using the HPLC assay, there was an average residual dose in the mouthpiece of 14.8%+4.3% and an average ED of 84.7%+3.1%, yielding a drug mass balance of 99.5%+2.0%. Based on this evaluation, HPLC was determined as a suitable method for determining the ED of the formulation.

### Example 5. Study of 6-Month Chemical Stability

The formulations of the present disclosure were investigated to evaluate physical and chemical stability at extended periods of time at 4° C. and at 25° C. A formulation sample solution was prepared containing 0.28 mg/mL exenatide (Bachem, Lot No. 1000004114), 10 mM sodium acetate trihydrate (USP, CAS No. 6131-90-4), and 140 mM mannitol (USP, CAS No. 69-65-8). The pH of sample solution was adjusted to 4.8±0.1 using 1.74 M glacial acetic acid (USP, CAS No. 64-19-7) and adjusted to a final volume of 250 mL in a sterile container. 3 mL of the sample solution was dispensed from the sterile container into individual serum vials (Wheaton®, 5 ml vial, Serum, Type I Clear Glass) that were plugged with a stopper (Wheaton®, Ultrapure straight plug stoppers) and sealed with aluminum (Wheaton®, 20 mm aluminum seal). Each of the serum vials containing the sample solutions were stored at 4° C. or at 25° C. in a stability chamber for the time periods indicated in Table 9.

Exenatide standard solutions of USP Exenatide RS (USP, Catalog No. 1269105) in water were also used in the RP-HPLC analysis.

The formulation samples were assessed as indicated in Table 9. For each time point, each serum vial containing a formulation sample was removed from its storage conditions and allowed to equilibrate to room temperature. The serum vials were observed for appearance and an aliquot taken for further analysis. For each aliquot, the pH was measured and then HPLC analysis was performed. The samples were analyzed neat for appearance and % recovery. The % recovery was based on the extenatide concentration (potency) at each time point vs. the extenatide concentration (potency) at T=0. The concentration of exenatide (major peak) and certain degrandants was determined after the HPLC. The % recovery is the normalization of the potency at each time point to facilitate observation of any change in potency over time. The extenatide concentration was the measurement of the major peak.

TABLE 9
Stability Assessment Parameters

| Storage | | |
|---|---|---|
| Conditions | Time Points (Months) | Testing |
| 4° C. | 0, 1, 3, 6 | Appearance, Potency, Exenatide- |
| 25° C. | 0, 1, 2, 3, 4, 5, 6 | Related Substances, and pH |

The chemical stability of the formulation after storage was determined by HPLC analysis. The samples were analyzed using two different reverse-phase high performance liquid chromatography (RP-HPLC) analytical methods to determine the concentration of exenatide in the formulation and main byproducts (i.e., exenatide-related substances, “ERS”). The first RP-HPLC analytical method utilized ammonium bicarbonate as the buffer (referred to herein as “the ammonium bicarbonate method”) and the second RP-HPLC utilized trifluoroacetic acid as a mobile-phase additive (referred to herein as “the TFA method”).

For the ammonium bicarbonate method, the HPLC analysis was conducted on a Waters) (Bridge C₁₈ column (inner diameter 3 mm, length 150 mm, 3.5 μm particle size packing, Part No. 186003028) at 60° C., eluted with Mobile Phase A and Mobile Phase B at a flow rate of 0.4 mL/min. Mobile Phase A was 10 mM ammonium bicarbonate in water (HPLC grade water) adjusted to pH 10 with ammonia solution (NH₄OH). Mobile Phase B was acetonitrile and Mobile Phase A at a ratio of 90:10. Mobile Phase A and B were eluted using the following elution gradient:

| | Mobile Phase A | Mobile Phase B |
|---|---|---|
| Time | (%) | (%) |
| 0 | 73 | 27 |
| 0.5 | 73 | 27 |
| 33 | 61 | 39 |
| 35 | 10 | 90 |
| 36 | 10 | 90 |
| 36.1 | 73 | 27 |
| 42 | 73 | 27 |

For the TFA method, the HPLC analysis was conducted on an Avantor® ACE® 5 μm C₁₈ column (inner diameter 3 mm, length 250 mm, 5 μm particle size packing, Avantor Performance Materials, Catalog No. ACE-221-2503) at 60° C., eluted with Mobile Phase A and Mobile Phase B at a flow rate of 0.55 mL/min at an injection volume of 30 μL. Mobile Phase A was 0.1% TFA (Thermo Scientific, Reference No. 28904) in HPLC grade water. Mobile Phase B was 0.1% TFA in a mixture of acetonitrile/water (both HPLC grade) at a ratio of 90:10. Mobile Phase A and B were eluted using the following elution gradient:

| | Mobile Phase A | Mobile Phase B |
|---|---|---|
| Time | (%) | (%) |
| 0 | 65 | 35 |
| 0.5 | 65 | 35 |
| 20.0 | 55 | 45 |
| 20.5 | 10 | 90 |
| 24.0 | 10 | 90 |
| 24.1 | 65 | 35 |
| 30.0 | 65 | 35 |

For the HPLC methods, in addition to exenatide as the major peak, exenatide-related substances (ERSes; i.e., degradants) formed in the formulation samples were characterized by their relative retention times (RRTs) with chromatograph peaks located at RRT 0.38, RRT 0.52, RRT 0.59, RRT 1.24, and RRT 1.86. RRT values can vary slightly from run to run. The response factor of the reference standard is related to its chromatographic area response by Equation (1) below:

$${R}_{f} = ( \frac{{A}_{std}}{{C}_{std}} ) , \qquad ( Eq .1 )$$

where R_{f} is the peak response factor relating the area of external reference standard peak to exenatide concentration in the reference standard; A_{std} is the area of exenatide peak in the standard; C_{std} is the exenatide concentration in the standard solution. The exenatide concentration in the sample is calculated by comparison to a single point external reference standard and is determined according to Equation (2):

$${C}_{Ex} = ( \frac{{A}_{Ex}}{{R}_{f}} ) , \qquad ( Eq .2 )$$

where A_{Ex} is the area of exenatide peak in the sample; C_{Ex} is the exenatide concentration in the sample solution. The percentage of exenatide in the sample solution was determined according to Equation (3) below:

$$\% Extenatide = ( \frac{{A}_{Ex}}{{A}_{S}} ) \times 100 , \qquad ( Eq .3 )$$

where A_{Ex} is the area of exenatide peak in the sample solution; Aₛ is the sum of total peak area (exenatide and all ERS peaks) in the sample solution. The percentage of individual ERSi in the sample solution was determined according to Equation (4) below:

$$\% ERSi = ( \frac{{A}_{i}}{{A}_{S}} ) \times 100 , \qquad ( Eq .4 )$$

where Aᵢ is the area of the ERSi peak in the sample solution; Aₛ is the sum of total peak area in the sample solution. The percentage of the total ERS in the sample solution was determined according to Equation (5) below

$$\% Total ERS = ( \frac{Σ {A}_{i}}{{A}_{S}} ) \times 100 , \qquad ( Eq .5 )$$

where Σ Aᵢ is the sum of area of all the ERS peaks in the sample solution; Aₛ is the sum of total peak area in the sample solution.

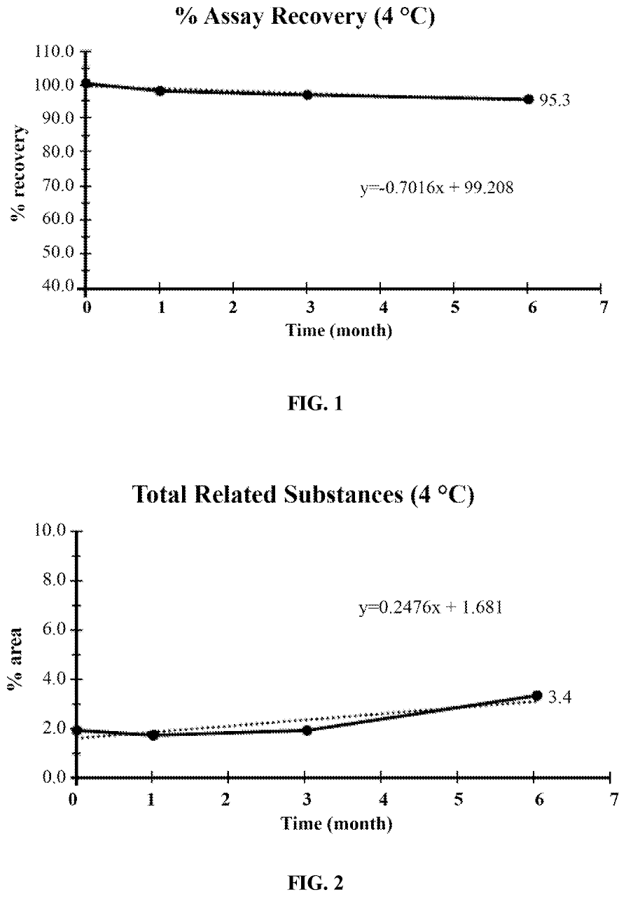

Table 10 provides the assay recovery values for formulation samples stored at 4° C. as analyzed according to the ammonium bicarbonate method. After 6 months, the formulation maintained a clear appearance indicating that it had good physical stability (e.g., the exenatide did not change physical state so as to precipitate out of solution). As shown in FIG. 1, the exenatide recovery for the formulation under storage conditions of 4° C. after 6 months is greater than 95% reflecting stable potency. Additionally, FIG. 2 shows that the formation of exenatide-related substances detected in the formulation after 6 months only accounted for 3.4% of the total peak area, which is well below the limit of impurities of 10%. From the slope of the linear regression shown in FIG. 2, the formulation samples had a degradation rate of about 0.25% ERS formation per month at 4° C. This suggests that after 24 months of storage at 4° C., the extrapolated degradation may be about 6.0% of total ERS. Thus, including the initial 2% amount of degradants present in the formulation at T=0, the projected total ERS at 24 months is 8.0%, which is below the maximum allowed 10% total impurity. Therefore, the formulation exhibited good chemical stability after 6 months for storage conditions at 4° C. with limited formation of byproducts.

TABLE 10
Formulation Stability at 4° C. - Ammonium Bicarbonate Method

| | Time (months) | | | |
|---|---|---|---|---|
| | 0 | 1 | 3 | 6 |
| Appearance | Clear | Clear | Clear | Clear |
| Assay (mg/ml) | 0.277 | 0.271 | 0.268 | 0.264 |
| % Assay Recovery | 100.0 | 97.8 | 96.6 | 95.3 |
| Exenatide Purity by RP-HPLC | 98.0 | 98.2 | 98.0 | 96.6 |
| Total ERS | 2.0 | 1.8 | 2.0 | 3.4 |
| ERS - RRT 0.38 | ND | ND | ND | ND |
| ERS - RRT 0.52 | 0.10 | 0.07 | 0.13 | 0.18 |
| ERS - RRT 0.59 | 0.64 | 0.79 | 1.01 | 0.96 |
| ERS - RRT 1.24 | 0.04 | 0.04 | 0.04 | D |
| ERS - RRT 1.86 | ND | ND | 0.04 | 0.04 |
| pH | 4.80 | 4.69 | 4.75 | 4.78 |

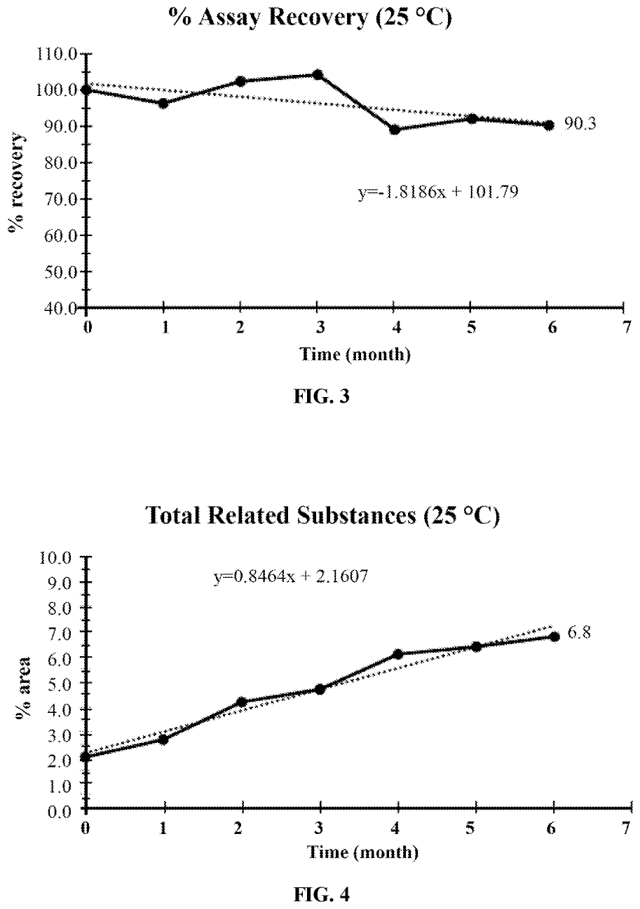

Table 11 provides the assay recovery values for formulation samples stored at 25° C. as analyzed according to the ammonium bicarbonate method. After 6 months, the formulation maintained a clear appearance indicating that it had good physical stability (e.g., the exenatide did not change physical state so as to precipitate out of solution). As shown in FIG. 3, the exenatide recovery for the formulation under storage conditions of 25° C. after 6 months is greater than 90% reflecting stable potency. Additionally, FIG. 4 shows that the formation of exenatide-related substances detected in the formulation after 6 months accounted for less than 7% of the total peak area, which is below the limit of impurities of 10%. Therefore, the formulation exhibited good stability after 6 months for storage conditions at 25° C. with limited formation of byproducts. From the slope of the linear regression shown in FIG. 4, the formulation samples had a degradation rate of about 0.85% ERS formation per month at 25° C. As a rule of thumb, the Arrhenius equation suggests that the reaction rate of a biological or chemical reaction doubles for every 10° C. Based on this, the 6 month stability of the formulation can be projected to 24 months stability, which is consistent with the extrapolated stability results from the above 4° C. stability study.

TABLE 11
Formulation Stability at 25° C. - Ammonium Bicarbonate Method

| | Time (months) | | | | | | |
|---|---|---|---|---|---|---|---|
| | 0 | 1 | 2 | 3 | 4 | 5 | 6 |
| Appearance | Clear | Clear | Clear | Clear | Clear | Clear | Clear |
| Assay (mg/ml) | 0.277 | 0.267 | 0.284 | 0.289 | 0.247 | 0.255 | 0.250 |
| % Assay Recovery | 100.0 | 96.3 | 102.4 | 104.2 | 89.1 | 92.1 | 90.3 |
| Exenatide Purity by | 98.0 | 97.3 | 95.8 | 95.3 | 93.9 | 93.6 | 93.2 |
| RP-HPLC | | | | | | | |
| Total Related | 2.0 | 2.7 | 4.2 | 4.7 | 6.1 | 6.4 | 6.8 |
| Substances | | | | | | | |
| ERS - RRT 0.38 | ND | 0.05 | 0.16 | 0.38 | 0.49 | 0.54 | 0.64 |
| ERS - RRT 0.52 | 0.10 | 0.22 | 0.41 | 0.58 | 0.73 | 0.86 | 0.94 |
| ERS - RRT 0.59 | 0.64 | 1.12 | 1.78 | 1.67 | 2.24 | 1.95 | 1.71 |
| ERS - RRT 1.24 | 0.04 | 0.02 | 0.03 | 0.01 | 0.02 | 0.04 | 0.08 |
| ERS - RRT 1.86 | ND | 0.14 | 0.30 | 0.38 | 0.43 | 0.50 | 0.60 |
| pH | 4.80 | 4.70 | 4.76 | 4.78 | 4.73 | 4.79 | 4.80 |

Based on the least squares regression of the values according to the ammonium bicarbonate method, the % assay recovery of the samples at 4° C. decreased at a rate of −0.70% per month, while the % assay recovery of the samples under accelerated storage conditions at 25° C. decreased at a rate of −1.82% per month. Thus, the formulation remained within stability specifications even at accelerated conditions for at least 6 months.

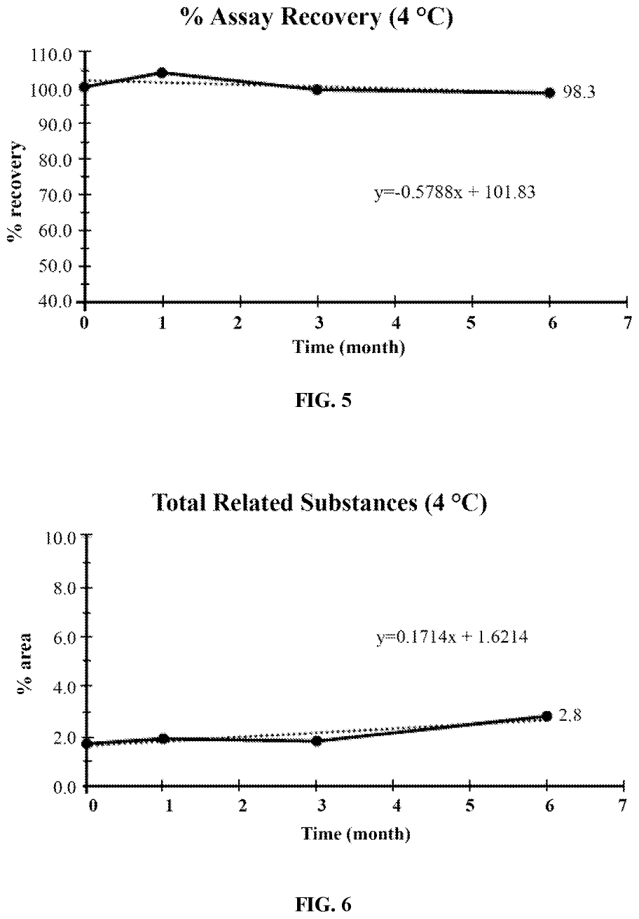

Table 12 provides the assay recovery values for formulation samples stored at 4° C. as analyzed according to the TFA method. After 6 months, the formulation maintained a clear appearance indicating that it had good physical stability (e.g., the exenatide did not change physical state so as to precipitate out of solution). As shown in FIG. 5, the exenatide recovery for the formulation under storage conditions of 4° C. after 6 months is greater than 98% reflecting stable potency. Additionally, FIG. 6 shows that the formation of exenatide-related substances in the formulation after 6 months accounted for less than 3% of the total peak area, which is well below the limit of impurities of 10%. From the slope of the linear regression shown in FIG. 6, the formulation samples had a degradation rate of about 0.17% ERS formation per month at 4° C. This suggests that after 2 years storage at 4° C., the extrapolated degradation could be 4.1% of total ERS. Thus, including the initial 1.7% amount of degradants present in the formulation at T=0, the projected total ERS at 24 months is 5.8%, which is below the maximum allowed 10% total impurity. Therefore, the formulation exhibited good stability after 6 months for storage conditions at 4° C. for storage conditions at 4° C. with limited formation of byproducts.

TABLE 12
Formulation Stability at 4° C. - TFA Method

Time (months)

| | 0 | 1 | 3 | 6 |
|---|---|---|---|---|
| Appearance | Clear | Clear | Clear | Clear |
| Assay (mg/ml) | 0.272 | 0.282 | 0.269 | 0.267 |
| % Assay Recovery | 100.0 | 104.0 | 99.2 | 98.3 |
| Exenatide Purity by | 98.3 | 98.1 | 98.2 | 97.2 |
| RP-HPLC | | | | |
| Total Related | 1.7 | 1.9 | 1.8 | 2.8 |
| Substances | | | | |
| ERS - RRT 0.59 | 0.56 | 0.60 | 0.82 | 0.61 |
| pH | 4.80 | 4.69 | 4.75 | 4.78 |

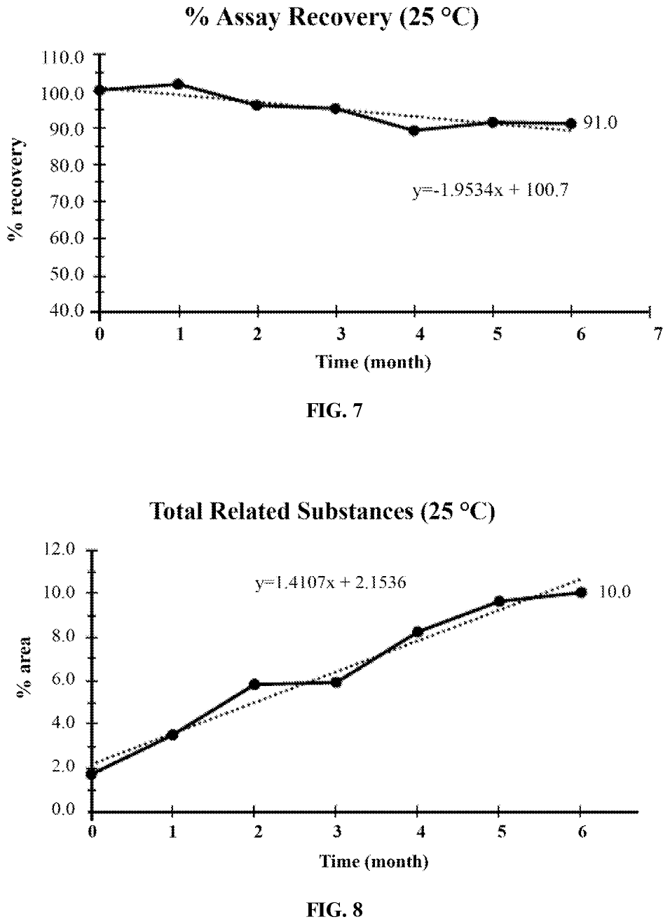

Table 13 provides the assay recovery values for formulation samples stored at 25° C. as analyzed according to the TFA method. After 6 months, the formulation maintained a clear appearance indicating that it had good physical stability (e.g., the exenatide did not change physical state so as to precipitate out of solution). As shown in FIG. 7, the exenatide recovery for the formulation under storage conditions of 25° C. after 6 months was 91%. Additionally, FIG. 8 shows that the formation exenatide-related substances detected in the formulation after 6 months accounted for 10% of the total peak area. Therefore, the formulation exhibited good stability after 6 months for storage conditions at 25° C. with limited formation of byproducts. Based on the Arrhenius equation, as discussed above, the 6 months stability can be projected to 24 months stability, which is consistent with the extrapolated stability result from the above 4° C. stability study. From the slope of the linear regression shown in FIG. 8, the formulation samples had a degradation rate of about 1.41% ERS formation per month at 25° C.

TABLE 13
Formulation Stability at 25° C. - TFA Method

| | Time (months) | | | | | | |
|---|---|---|---|---|---|---|---|
| | 0 | 1 | 2 | 3 | 4 | 5 | 6 |
| Appearance | Clear | Clear | Clear | Clear | Clear | Clear | Clear |
| Assay (mg/ml) | 0.272 | 0.276 | 0.260 | 0.258 | 0.242 | 0.248 | 0.247 |
| % Assay Recovery | 100.0 | 101.7 | 95.9 | 95.0 | 89.1 | 91.3 | 91.0 |
| Exenatide Purity by | 98.3 | 96.5 | 94.2 | 94.1 | 91.8 | 90.4 | 90.0 |
| RP-HPLC | | | | | | | |
| Total Related | 1.7 | 3.5 | 5.8 | 5.9 | 8.2 | 9.6 | 10.0 |
| Substances | | | | | | | |
| ERS - RRT 0.59 | 0.56 | 0.86 | 1.48 | 1.25 | 1.95 | 1.28 | 0.95 |
| pH | 4.80 | 4.69 | 4.76 | 4.78 | 4.73 | 4.79 | 4.80 |

Based on the least squares regression of the values according to the TFA method, the % assay recovery of the samples at 4° C. decreased at a rate of −0.57% per month, while the % assay recovery of the samples under accelerated storage conditions at 25° C. decreased at a rate of −1.95% per month. Thus, the assay remained within stability specifications even at accelerated conditions for at least 6 months.

In comparison to the ammonium bicarbonate method, the results of the TFA method detected a higher total % exenatide-related substances in the assay. The TFA method results also show a good, non-distorted baseline around the main exenatide peak relative to the ammonium bicarbonate method (data not shown), which can enable better data integration and assessment of impurities. This may indicate that the TFA method is more suitable for sample analysis. An ion exchange method may also be used to monitor specific impurity peaks that do not separate well by RP-HPLC.

The results of the study show that the inhalation formulation is stable for at least 6 months at 25° C. Using the ammonium bicarbonate method, a total of 3.4% and 6.8% exenatide-related substances were detected for samples at 4° C. and 25° C., respectively, at 6 months. Similarly, using the TFA method, a total of 2.8% and 10.0% exenatide-related substances were detected for samples at 4° C. and 25° C., respectively, at 6 months. Both sets of values remain within specifications. For storage conditions at 4° C., exenatide-related substances were produced at a rate of approximately 0.2% per month, which indicates that the formulation could provide at least 40 months of stability before reaching the impurities limit of 10%, based on the average of the slope of the linear regression plots at 4° C. for the ammonium bicarbonate method and the TFA method. The performance of the formulation at 25° C. also suggests that the formulation has at least 2 years of stability at 4° C. before reaching the impurities limit of 10% based on the Arrhenius law, based on the slope of the linear regression plot at 25° C. The Arrhenius equation gives a “rule of thumb” that a 10° C. temperature rise doubles most biological and chemical reaction rates. The production of impurities in the formulation occurs via multiple chemical reactions, as evidenced by the multiple degradation products. Each chemical reaction has its own reaction rate and, in aggregate, they reflect a composite reaction rate for the formulation. It is a reasonable expectation that the composite reaction rate would follow the Arrhenius law.

The foregoing description of certain embodiments has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure. Certain features that are described in this specification in the context of separate embodiments can also be implemented in combination in a single implementation. Conversely, various features that are described in the context of a single implementation can also be implemented in multiple ways separately or in any suitable subcombination. Moreover, although features may be described above as acting in certain combinations, one or more features from a combination can in some cases be excised from the combination, and the combination may be directed to a subcombination or variation of a subcombination. Thus, particular embodiments have been described. Other embodiments are within the scope of the disclosure. All printed patents and publications referred to in this application are hereby incorporated herein in their entirety by this reference.

What is claimed is:

1. A pharmaceutical composition comprising exenatide, or a pharmaceutically acceptable salt thereof, and an aqueous buffer, wherein the pharmaceutical composition is packaged for administration via inhalation.

2. The composition of claim 1, wherein the concentration of exenatide or the pharmaceutically acceptable salt thereof ranges from about 200 μg/mL to about 800 μg/mL.

3. The composition of claim 1, wherein the pH of the composition ranges from about 4.6 to about 5.2.

4. The composition of claim 1, wherein the aqueous buffer comprises sodium acetate.

5. The composition of claim 4, wherein the concentration of sodium acetate ranges from about 5 mM to about 50 mM.

6. The composition of claim 1, wherein the osmolarity of composition ranges from about 50 mOsm to about 400 mOsm.

7. The composition of claim 1, wherein the pharmaceutical composition additionally comprises mannitol.

8. The composition of claim 7, wherein the concentration of mannitol ranges from about 50 mM to about 200 mM.

9. The composition of claim 1, wherein the pharmaceutical composition is substantially free of preservatives, stabilizers, and/or surfactants.

10. The composition of claim 1, wherein:
the exenatide or the pharmaceutically acceptable salt thereof is present in an amount ranging from about 250 μg/ml to about 350 μg/ml;
the pH of the aqueous buffer is ranges from about 4.7 to about 4.9;
the osmolarity of the composition ranges from about 150 mOsm to about 200 mOsm; and
the composition is substantially free of preservatives.

11. The composition of claim 1, wherein the pharmaceutical composition is packaged in a dispenser for administration via inhalation using a vibrating mesh nebulizer.

12. The composition of claim 1, wherein the composition comprises exenatide acetate.

13. The composition of claim 1, wherein the composition has a chemical stability of at least 95% for 6 months under storage conditions of 4° C.

14. A method of treating a subject with diabetes mellitus, comprising administering a therapeutically effective amount of the pharmaceutical composition according to claim 1, wherein the composition is administered to the subject via inhalation.

15. The method of claim 14, wherein the composition is administered using a vibrating mesh nebulizer.

16. The method of claim 14, wherein the therapeutically effective amount of the pharmaceutical composition is administered in one to five breaths.

17. The method of claim 14, wherein the pharmaceutical composition is administered two times per day or three times per day.

18. The method of claim 14, wherein administering the pharmaceutical composition comprises aerosolizing one to six drops of the pharmaceutical composition.

19. The method of claim 18, wherein the volume of each drop ranges from about 20 μL to about 60 μL.

20. The method of claim 14, wherein the exenatide or the pharmaceutically acceptable salt thereof is present in an amount ranging from about 200 μg/mL to about 800 μg/mL, and wherein administering the composition comprises aerosolizing the composition at a rate ranging from 350 μL/min to about 700 μL/min.

21. The method of claim 14, wherein 1-15 μg of exenatide or a pharmaceutically acceptable salt thereof is delivered to the lungs of the subject in each administration.
